(19) United States
(12) Patent Application Publication
(10) Pub. No.: US 2007/0128094 A1
(43) Pub. Date: Jun. 7, 2007

(54) Microbial barrier system having a plurality of sterilization sheets

(75) Inventors: Agnes Paris-Jolly, Issy Les Moulineaux (FR); Christophe Simon, Issy Les Moulineaux (FR)

Correspondence Address: BIRCH STEWART KOLASCH & BIRCH, PO BOX 747, FALLS CHURCH, VA, 22040-0747, US

(73) Assignee: ARJO WIGGINS

(21) Appl. No.: 11/292,061

(22) Filed: Dec. 2, 2005

(51) Int. Cl. A61L 2/00

(52) U.S. Cl. 422/292; 53/466

(57) ABSTRACT

A microbial barrier system (MBS) for medical use having two or more sheets. The MBS includes an outer sterilization sheet of wet-laid paper or nonwoven formed from cellulosic fibers and an inner sterilization sheet of SMS. The two sheets are in registration with each other without bonding. The sterilization sheets may be provided alternatively in a stack to provide a simple arrangement for using the wraps.

### BACKGROUND OF THE INVENTION

### Field of the Invention

The present invention relates generally to a medical microbial barrier system and more particularly to a two or more sheet non-bonded medical microbial barrier system which is sterilized for making a sterile barrier system.

### DISCUSSION OF THE BACKGROUND

International Standard ISO 11607-1 defines a sterile barrier system as a minimum package that prevents ingress of microorganisms and allows aseptic presentation of the product at the point of use.

In medical situations, and especially in operating rooms, it is imperative that medical devices which have been sterilized remain sterile until they are used. One common method of achieving this has been to wrap the medical devices in some type of material in a prescribed fashion in order to prevent microorganisms (e.g. bacteria, virus, spores . . . ) from easily gaining access to the devices. Since the instruments are often wrapped before the sterilization process, it is necessary that the wraps allow the sterilizing agent, such as steam or a sterilization gas, to easily gain access to the medical devices while preventing access for microorganisms.

In the past, linen was used for this purpose. The linen had the ability to be washed and reused. However, in order to lower infection rates, many hospitals now use single-use wraps.

Wrapping sheets are now made from various materials including woven and nonwoven sheets. These particular sheets may be made of various materials in order to provide a number of different properties. One property of particular interest is the ability to prevent penetration of microorganisms through the packaging. Another property is the ability to avoid being ripped or torn while being handled. Another property is the avoidance of being wetted by various liquids which may be spilled, since this can provide a quicker path for microorganism entry. Likewise, other properties are desirable in these materials. Particular methods of wrapping the instrument tray in the sheets are well defined (e.g. Good hospital practice: Steam Sterilization and Sterility Assurance—A.A.M.I.S.S.S.A.—1988). It is common to use a method of double wrapping. One method is a sequential wrapping by wrapping for example an instrument tray in a first sterilization sheet which is then wrapped again by another sterilization sheet.

Some systems have attempted to avoid the step of the second folding by providing a double layer of bonded wrap sheets for a simultaneous wrapping. Accordingly, a single folding step allows for a two layered wrapping in this process. This simplifies the wrapping process and also makes it easier to unwrap the instruments when needed in the operating room.

Various materials have now been used to form these sheets. For example, PCT Publication WO 95/01135 describes a two-layer wrap system where both layers are formed from a synthetic polymeric laminate dry-laid nonwoven material known as SMS (Spunbond/Meltblown/Spunbond). This material is made from 3 separate layers which are laminated together. In general, any of the layers may be replaced with 2 or more layers of the same material. Thus, SMS is equivalent to SSMS, SMMS, SMMMS, etc. The central layer is one or more plys of meltblown layer made from extruded polyolefin fibers having a fine fiber structure which permits the sterilizing agent to pass therethrough while preventing the passage of microorganisms. The outer two layers are one or more plys of spunbond material from continuous extruded polyolefin fibers laid down in a random pattern and then thermobonded to each other. The meltblown fibers are generally of a smaller diameter and have a more discontinuous length than the fibers in the spun bonded layers. The spunbond layers provide a greater portion of the strength factor for the laminate. In this publication, the two layers of SMS are bonded together along two edges so that the wrap may be more easily handled during the wrapping and unwrapping process.

U.S. Pat. No. 6,630,104 shows another two layer sterilization wrap for medical instruments. In this arrangement, a single sheet of SMS in the shape of an elongated rectangle is folded so as to form a two layer structure. The two layers have been bonded together in order to retain this configuration.

Another sterilization wrap is described in U.S. printed application 2005/0008527. (See FIG. 1) In this arrangement, two layers of SMS are used in register with one another and joined by bonding along 4 edges.

While these and other systems have been successful in providing the necessary functions for a sterilization wrap, further improvements are always welcome in arrangements which decrease the penetration of microorganisms through the wrap while allowing easier handling both before the sterilization and in the process of opening the pack by unwrapping the outer sheet before the operating room and the sterile sheet in the operating room.

### SUMMARY OF THE INVENTION

The present invention provides an improved microbial barrier system (MBS) for sterilization.

The present invention further provides an improved two sterilization sheet wrap.

The present invention still further provides a microbial barrier system which includes an outer sheet of cellulosic material and an inner sheet of SMS (spunbond/meltblown/spunbond). As mentioned above, in the present invention, it is intended that SMS is equivalent to SSMS, SMMS, SMMMS, etc.

The present invention also provides an improved two sheet sterilization packaging without bonding between the two sheets.

The present invention still further provides an improved two sheet sterilization packaging where an outer sheet of cellulosic material and an inner SMS sheet overlay each other in registration without bonding.

The present invention still further provides for a simplified sterilization wrap presentation where a stack of wrap sheets may be provided with alternate sheets of cellulosic material and SMS so that two sheets may be removed together simply.

The present invention also provides for an improved method of providing a two sheet sterilization wrap.

The present invention still further provides a method for providing a two sheet sterilization wrap by unrolling a reel of a first material web and unrolling a reel of a second material web, rolling together the two material webs and cutting two sheets which overlay each other in registration.

The present invention provides these advantages by providing a double sheet MBS having an outer sheet of cellulosic material and an inner sheet of SMS where the two sheets are overlaying each other in registration but are not bonded together.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a perspective view of prior art sterilization wrap system;

FIG. 2 is a perspective view of the sterilization wrap of the present invention; and

FIG. 3 is a perspective view showing a stack of sterilization wraps according to the present invention.

### DETAILED DESCRIPTION OF THE PREFERRED EMBODIMENTS

The present invention will become more fully understood from the detailed description given hereinbelow and the accompanying drawings, which are given by way of illustration only, and thus are not limitative of the present invention, and wherein:

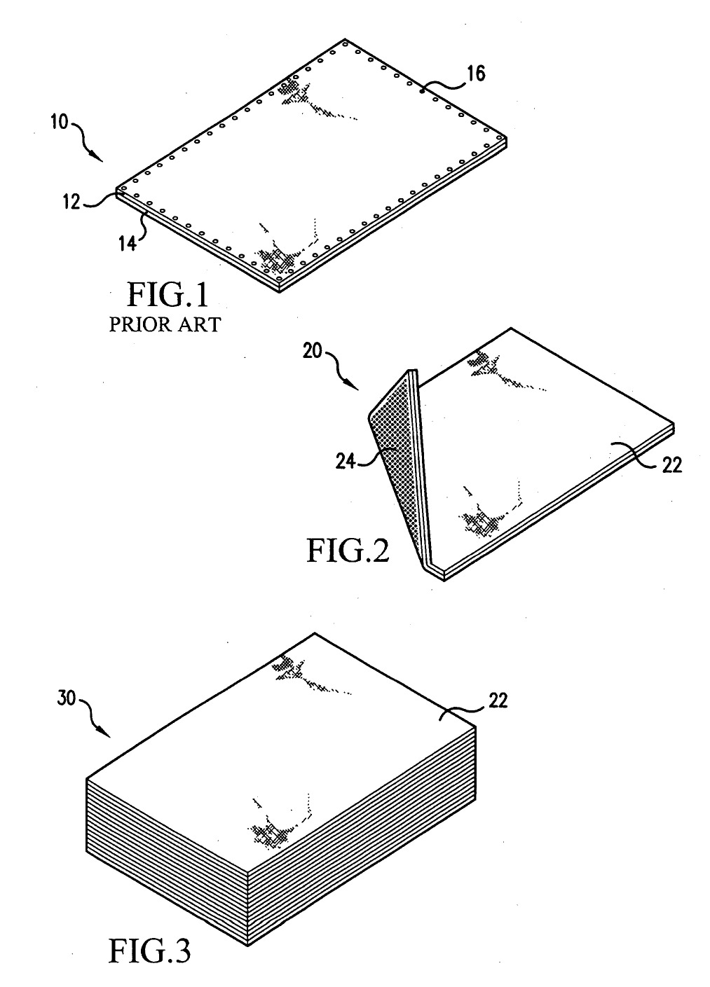

FIG. 1 shows a perspective view of a prior art arrangement such as described above. A two layer sterilization wrap 10 is shown, where the two layers 12, 14 are bonded together 16 in order to simplify the handling process.

The present invention 20 utilizes two sheets of differing material without being bonded, as shown in FIG. 2. In particular, Applicants have noted that the use of a cellulosic sheet 22 containing, preferably at least 30% of cellulose fibers for the total composition of fibers, the remainder of the fibers being synthetic fibers, in particular polyester fibers, produces advantageous results. These cellulosic sheets can be wet-laid sheets or dry-laid sheets such as hydroentangled or thermobonded nonwovens. One such sheet can be a sheet sold in the Sterisheet® range. In particular, the use of such a cellulosic sheet as the outer sheet improves the feel of the product and provides additional microbial barrier performance. The inner sheet 24 is SMS.

Compared to SMS, the person handling the cellulosic sheet notices a softer feel through the cellulosic sheet as compared to SMS. Furthermore, SMS is oil based and leaves a residue on the user's hands. Further, it is common for users to use hand lotions due to the dryness and sensitivity problems as a result of continuous contact with SMS, which is not necessary for cellulosic sheets. Also, it has been found that the cellulosic sheet folds, creases and conforms better than SMS, by having a better memory for the folds.

Perhaps more importantly, the uniform texture of the cellulosic sheet allows for easy detection of pinhole sized defects. When the sterilization pack is opened, the wrap is inspected to see if pinhole defects can be found. This is difficult, or even impossible, for SMS since it is thermo-calendered and so embossed which makes the defects hard to see. Applicants have found that pinholes in cellulosic material are much more easily detected, which provides an additional safety feature to detect direct passages through which microorganisms can easily enter.

Also, Applicants have noted that the hand lotions that may be on the hands of sterilization services nurses destroy the water and/or alcohol repellency of an SMS sheet. By using a cellulosic sheet on the outside, this does not happen. Also, the cellulosic sheet has an additional antistatic effect because it has a higher electrical conductivity, due to cellulose content, than SMS, which is 100% synthetic. This antistatic effect avoids the electrostatic attraction of dust which is a transmitting vehicle for microorganisms. When SMS is used as the outer layer, it must receive a specific additive to be alcohol repellent, which is antagonistic to the antistatic treatment. Furthermore, these treatments are expensive and affect the total cost of the sterilization packaging.

Further, Applicants have discovered that a wet pack effect is reduced when the cellulosic sheet forms the outer sheet of the packaging. This wet pack effect is the condensation of water within the pack after sterilization. Since the wetness on the inside of the pack can effect sterility, this is undesirable.

Applicants have also discovered that if the two sheet wrap is formed with one sheet of cellulosic material on the outside and an inner sheet of SMS that bonding with an adhesive or thermal bond between the two sheets is unnecessary. There is a certain affinity between adjoining sheets of these materials that cause them to couple without bonding. As a result, it is not necessary to have any weak points on the sheet such as for bonding which allow microorganism penetration. At the same time, these sheets overlay in registration so as to be easy to handle when wrapping and unwrapping. Further, the SMS sheet provides the higher mechanical strength for the MBS and the cellulosic sheets provide a high microbial barrier.

The outer sheet may be a sheet obtained by a papermaking route (wet laid process), comprising only cellulose fibers, the fibers possibly being modified, such as rayon fibers resulting from the sodium hydroxide treatment of viscose or regenerated cellulose fibers in solvent medium, such as those sold under the brand names LYOCELL® or TENCEL®, as a mixture with a wet strength agent and a paper cohesive agent added into the bulk or on the surface, such as a polyvinyl alcohol, a starch or a polymer added in the form of a stable aqueous emulsion, especially acrylic polymers or acrylates.

The outer sheet may also be a sheet, obtained by a wet-laid or dry-laid process comprising cellulose fibers, possibly modified like the rayon fibers coming from the sodium hydroxide treatment of viscose, or regenerated cellulose fibers in solvent medium, such as those sold under the trademarks LYOCELL® or TENCEL®, as a mixture with synthetic fibers, all these fibers being bonded either by thermal bonding or by water-jet bonding (i.e. hydroentangling), or chemically by means of the addition in bulk, or by means of a surface treatment such as in a sizing press or by spraying, of a binder normally used in papermaking such as a polyvinyl alcohol, a starch or a polymer added in the form of a stable aqueous emulsion. Preferably, the synthetic fibers are in amounts ranging between 5 and 70 parts by dry weight, more preferably between 5 and 50 parts, the total sum of the fibers making 100 parts.

The sheets may optionally be creped, microcreped or embossed. They may be colored or tinted.

Preferably, the synthetic fibers are chosen from fibers of homopolymers or copolymers of olefins, of polyester, of polyamide and blends thereof. These fibers may also be two-component fibers having a core and a shell differing in chemical nature and/or having different properties, such as, for example, their melting points. These fibers may be chopped fibers. The synthetic fibers can be heat fusible as well.

The synthetic fibers preferably have a length ranging between 1 and 30 mm and, moreover, a linear density (mass per unit length) ranging between 0.4 and 5 dtex.

The sheet is produced on a Fourdrinier paper machine or an inclined wire device. For example, cellulose fibers and polyester synthetic staple fibers in respective proportions of 75 parts and 25 parts by dry weight are put into suspension in aqueous medium. The cellulose fibers are a mixture of 20% by weight of short fibers and a balance to 100 of long fibers, the fibers being refined to 25° SR. The polyester fibers have a length ranging between 5 and 25 mm and a linear density of 1.7 dtex. Added to this suspension are 0.26% by dry weight of the total composition of the sheet of a wet-strength agent of the PAE (polyamine epichlorohydrin) and 1% by dry weight of the total composition of the sheet of a cationic starch as internal cohesion agent. This suspension is dewatered on the wire of the machine in order to form the sheet.

The sheet is impregnated in a sizing press with an acrylic synthetic cohesion agent introduced in the form of a stabilized aqueous emulsion. This acrylic agent is present in an amount of 14 g/m² by dry weight. The sheet is dried at about 120° C. The sheet then has a grammage of 78 g/m². The acrylic is chosen so as to provide water repellency characteristics. A fluorocarbon resin can also be added to provide alcohol repellency to the sheet.

As example of sheets in registration, the following sheets can be used:

the cellulosic sheet can be a wet laid paper STERISHEET® 88 having a grammage of 78 g/m², a tear strength of 1750 mN in Machine Direction and of 2600 mN in Cross Direction, measured according to European standard EN21974, an average Bacterial Filtering Efficiency of 94% measured according to ASTM-F-2101 standard, and an electrical resistivity of 1.10¹² ohms/square according to the BS 6524 standard at a relative humidity of 40%

the SMS can be a sheet having a grammage of 47 g/m², a tear strength of 5500 mN in Machine Direction and of 9500 mN in Cross Direction, measured according to European standard EN 21974, an average Bacterial Filtering Efficiency of 79.1%, measured according to ASTM-F-2101 standard, and an electrical resistivity of 3.10¹⁴ ohms/square according to the BS 6524 standard at a relative humidity of 40%.

The resulting system of these two sheets has a Bacterial Filtering Efficiency of 98.5%.

Furthermore, it is possible to provide these sterilization sheets in a multiple wrap stack 30 (See FIG. 3) having a great many wraps, such as perhaps 100. Of course, different numbers can be used from several to several hundred. By having the wraps in a stack where the interleaved stack is alternate sheets of SMS and cellulosic material, the user can place the instrument tray or medical device on top of the stack, merely thumb the edge of the stack to pull off the top two sheets, and fold the wrap around the instrument. After the package is removed, the next instrument tray or medical device may be placed on the top sheet of the stack. This allows for a simple operation of providing wraps to a number of instrument trays or medical devices.

The stack of interleaved sheets can be formed by unrolling a reel of a web of the cellulosic material and also unrolling a reel of a web of SMS. The two webs are then rolled together. The combined webs are then unrolled and cut into sets of two sheets which overlay each other in registration. The size of the set would be appropriate to their use for wrapping medical devices. The sets are then stacked to form the multiple wrap stack 30 shown in FIG. 3.

As noted above, the present invention includes a cellulosic sterilization sheet acting as an outside sheet and an SMS sheet acting as an inside sheet of a MBS having a plurality of sheets. It should be noted that even if these two sheets are reversed, many of the favorable features would be retained. However, some properties would not be present including those that rely on the feel of the cellulosic sheet to be on the outside. Thus, the difference in feel, the inertia against hand cream, the easy detection of pinhole defects, the natural antistatic effect and the alcohol repellency effect is lost if the layers are reversed. Accordingly, the preferable arrangement is the use of the cellulosic material as the outside sheet and the SMS sheet as the inner layer. While two sheet systems have been described, it is also possible to use a system of three or more sheets for additional protection.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A microbial barrier system having a plurality of sheets comprising:
an outer sheet made of a cellulosic material;
an inner sheet being a SMS sheet;
said outer sheet and said inner sheet overlaying each other in registration without bonding.

2. The system according to claim 1, wherein the cellulosic material is a wet-laid paper or a nonwoven comprising cellulosic fibers.

3. The system according to claim 1, wherein said SMS sheet is a multi-layer laminate having outer layers formed from one or more plys of spun-bond extruded polyolefin fibers and a center layer formed from one or more plys of extruded polyolefin fibers of a smaller diameter and a more discontinuous length than the outer layers.

4. The system according to claim 2, wherein the cellulosic material is made of cellulosic fibers of at least 30% in dry weight of total weight of fibers.

5. The system according to claim 4, wherein the cellulosic material is made of cellulosic fibers of at least 50% in dry weight of total weight of fibers.

6. A kit for providing sterilization wraps, comprising:
a stack of interleaved sheets, said sheets alternating between a first material and a second material, the outer sheet of the two sheets being a cellulosic material and the inner sheet being a SMS sheet, the two sheets overlaying each other in registration without bonding to form a two sheet wrap.

7. The kit according to claim 6, wherein the cellulosic material is a wet-laid paper or a nonwoven comprising cellulosic fibers.

8. The kit according to claim 6, wherein the SMS sheet is a multi-layer laminate having the outer layers formed from one or more plys of spun-bond extruded polyolefin fibers and the center layer formed from one or more plys of extruded polyolefin fibers of a smaller diameter and a more discontinuous length than the outer layers.

9. The kit according to claim 7, wherein the cellulosic material comprises cellulosic fibers of at least 30% in dry weight of total weight of fibers.

10. The kit according to claim 9, wherein the cellulosic material comprises cellulosic fibers of at least 50% in dry weight of total weight of fibers.

11. A method for providing a two sheet sterilization wrap comprising the steps of:
providing a stack of interleaved sheets, the stack having a plurality of sets, each set having a sheet of a cellulosic material and a sheet of SMS, the sheets of each set overlaying each other in registration without bonding;
removing a set of sheets from the stack to provide a sterilization wrap, the set of sheets including a cellulosic material sheet and sheet of SMS; and
using the cellulosic material as an outer layer in the sterilization wrap.

12. A method for providing a two sheet sterilization wrap according to claim 11 comprising, before providing a stack of interleaved sheets, the steps of:
unrolling a reel of a web of the cellulosic material and unrolling a reel of a web of SMS;
rolling together the two webs;
unrolling and cutting the two webs in two sheets overlaying each other in registration, at a size appropriate to their use, to form a set;
stacking said sets.

13. The method according to claim 11, wherein the SMS is a multi-layer laminate having outer layers formed from one or more plys of spun-bond extruded polyolefin fibers and a center layer formed from one or more plys of extruded polyolefin fibers of a smaller diameter and a more discontinuous length than the outer layers.

14. The method according to claim 11, wherein the cellulosic material is a wet-laid paper or a nonwoven comprising cellulosic fibers.

15. The method according to claim 11, wherein the cellulosic material comprises cellulosic fibers of at least 30% in dry weight of total weight of fibers.

16. The method according to claim 15, wherein the cellulosic material comprises cellulosic fibers of at least 50% in dry weight of total weight of fibers.

17. The method according to claim 11, further comprising the step of wrapping medical devices or tray in the set of sheets.

18. A sterile barrier system, comprising:
an outer sheet made of a cellulosic material;
an inner sheet being a SMS sheet;
the said outer sheet and the said inner sheet overlaying each other in registration without bonding.
